(19) United States
(12) Patent Application Publication
(10) Pub. No.: US 2008/0027105 A1
(43) Pub. Date: Jan. 31, 2008

(54) Dosing Regimen of Flavopiridol for Treating Cancer`

(75) Inventors: Jose-Ramon SUAREZ, Neshanic Station, NJ (US); John BYRD, Columbus, OH (US); Michael GREVER, Powell, OH (US); James DALTON, Columbus, OH (US); Thomas LIN, Columbus, OH (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignees: AVENTIS PHARMACEUTICALS INC., Bridgewater, NJ (US); OHIO STATE UNIVERSITY RESEARCH FOUNDATION, Columbus, OH (US)

(21) Appl. No.: 11/841,241

(22) Filed: Aug. 20, 2007

Related U.S. Application Data
(63) Continuation of application No. PCT/US2006/009162, filed on Mar. 15, 2006.
(60) Provisional application No. 60/662,503, filed on Mar. 16, 2005.

(51) Int. Cl. A61K 31/452

(52) U.S. Cl. 514/320

(57) ABSTRACT

A dosing regimen comprising a therapeutically effective amount of flavopiridol or a pharmaceutically acceptable salt thereof optionally in combination with a pharmaceutically acceptable carrier suitable for bolus infusion and subsequently for infusion is disclosed and claimed. Also disclosed and claimed is a method of treating a variety of cancers in a patient comprising administering to said patient the dosing regimen of the invention.

### BACKGROUND OF THE INVENTION

1. Field of the Invention

The present invention relates to a dosing regimen of flavopiridol for treating cancer. More specifically, this invention relates to a dosing regimen of flavopiridol for treating chronic lymphocytic leukemia (CLL) among various other diseases.

2. Description of the Art

Cyclin-dependent kinases (CDKs) are important regulators that control the timing and coordination of the cell cycle. CDKs form reversible complexes with their obligate cyclin partners to control transition through key junctures in the cell cycle. For example, the activated CDK4-cyclin D1 complex controls progression through the G1 phase of the cell cycle, while the CDK1-cyclin B1 complex controls entry into the mitotic phase of the cell cycle. Endogenous cyclin dependent kinase inhibitory proteins (CDKIs) are known that bind either the CDK or cyclin component and inhibit the kinase activity. In many tumors such as melanomas, pancreatic and esophageal cancers, these natural CDKIs are either absent or mutated. Thus, selective CDK inhibitors may prove to be effective chemotherapeutic agents.

Flavopiridol, (cis-5,7-dihydroxy-2-(2-chlorophenyl)-8-[4-(3-hydroxy-1-methyl)-piperidinyl]-1-benzopyran-4-one) hydrochloride, of formula (I) is a synthetic flavone that has been shown to have antitumor activity against various tumor cells lines, such as human lung carcinoma and breast carcinoma. It also inhibits tumor growth in xenograft models. It has been shown to induce arrest in both the G1 and G2 phases of the cell cycle. Flavopiridol is a potent and selective inhibitor of the CDKs, and its antitumor activity is related to its CDK inhibitory activity. Studies have shown that its tumor cell growth inhibitory activity occurs in a cell cycle specific manner. See Bioorg. & Med. Chem. Letters 10:1037-1041 (2000).

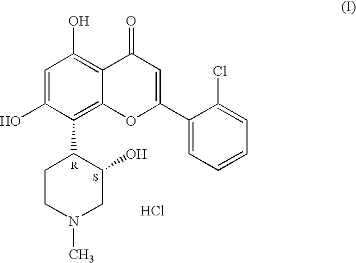

Despite promising pre-clinical studies with the cyclin-dependent kinase inhibitor flavopiridol in relapsed chronic lymphocytic leukemia (CLL) and other diseases, previous clinical trials with this agent have been disappointing. The discovery of high protein binding of flavopiridol in human serum relative to fetal bovine serum may have misdirected the schedule of administration and development of this agent.

Chronic lymphocytic leukemia (CLL) is one of the most common types of adult leukemia. Several therapeutic advances have occurred for the treatment of CLL over the past decade including the introduction of the purine analog fludarabine and combination strategies with alkylator therapy and/or monoclonal antibodies such as rituximab. See for example, Byrd J. C. et al., “Chronic lymphocytic leukemia. Hematology,” Am Soc Hematol Educ Program, 2004:163-83. However, treatment strategies for this disease are generally not curative outside of allogeneic stem cell transplant with virtually all patients relapsing and becoming resistant to fludarabine. In addition, dramatic anti-tumor responses and acute tumor lysis syndrome (TLS) commonly observed in curable acute leukemias and lymphomas are only rarely observed in CLL with any of the therapies currently used to treat this disease.

Studies examining the genetic features of fludarabine-refractory CLL have identified a high (42-50%) frequency of deletion and/or mutation of the p53 gene (see, for example, Sturm I. et al., “Mutation of p53 and consecutive selective drug resistance in B-CLL occurs as a consequence of prior DNA-damaging chemotherapy,” Cell Death Differ 2003; 10:477-84; and Lozanski G. et al., “Alemtuzumab is an effective therapy for chronic lymphocytic leukemia with p53 mutations and deletions,” Blood 2004; 103:3278-81) as compared to symptomatic, untreated patients (7-10%). See, for example, Thornton P. D., et al., “Characterization of TP53 abnormalities in chronic lymphocytic leukemia,” Hematol J. 2004; 5:47-54; and Oscier D. G., et al., “Multivariate analysis of prognostic factors in CLL: clinical stage, IGVH gene mutational status, and loss or mutation of the p53 gene are independent prognostic factors,” Blood 2002; 100:1177-84. The p53 mutation and/or deletions are predictive of treatment failure with alkylator, fludarabine, and rituximab-based therapies. At the present time only alemtuzumab (Campath-1H) or high dose methylprednisolone have efficacy in this highly resistant molecular subset of CLL. See for example Lozanski et al. as referred to hereinabove as well as Stilgenbauer S., et al. “Campath-1H in refractory CLL-complete remission despite p53 gene mutation,” Blood 2001; 98; and Thornton P. D. et al., “High dose methylprednisolone can induce remissions in CLL patients with p53 abnormalities,” Ann. Hematol. (2003) 82:759-765. However, these treatments are quite immunosuppressive and can result in life-threatening complications. In addition, patients with fludarabine-refractory have a suspected survival of 10-13 months, approximating that observed in acute leukemia. See, Keating M. J., et al., “Results of first salvage therapy for patients refractory to a fludarabine regimen in chronic lymphocytic leukemia” Leuk. Lymphoma 2002; 43:1755-62; and Perkins J. G., et al., “Frequency and type of serious infections in fludarabine-refractory B-cell chronic lymphocytic leukemia and small lymphocytic lymphoma: implications for clinical trials in this patient population,” Cancer 2002; 94:2033-9. Developing new therapies for patients with CLL, particularly those with fludarabine-refractory disease or p53 mutations and/or deletions, remains a very high priority.

It has now been found, and this forms the subject of the present invention, that the efficacy of flavopiridol or its analogs can be considerably improved when they are administered using a dosing regimen which involves initial bolus administration of flavopiridol followed by an infusion for a limited period of time, said regimen being repeated as needed for the treatment of cancers such as refractory CLL.

All of the references described herein are incorporated herein by reference in their entirety.

### SUMMARY OF THE INVENTION

Surprisingly, it has now been found that a dosing regimen comprising first a bolus infusion for a short duration of time and a subsequent infusion of flavopiridol, including any of its pharmaceutically acceptable salts, stereoisomers, enantiomers or a suitable analog thereof, remarkably improves efficacy of the drug in treating a patient suffering from a variety of cancers. Thus in accordance of this invention there is provided a dosing regimen comprising:
a) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
b) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

In another aspect of this invention there is also provided a method of treating a cancer selected from the group consisting of chronic lymphocytic leukemia (CLL), acute lymphocytic leukemia (ALL), lymphoma, acute leukemia, esophageal cancer, solid tumors, breast cancer, lung cancer and prostate cancer in a patient comprising administering to said patient a dosing regimen comprising:
a) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
b) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

Various other aspects of this invention will become apparent from the detailed discussion of the invention that follows.

### BRIEF DESCRIPTION OF THE DRAWINGS

FIG. 1 shows progression-free survival in patients with previously treated CLL treated with flavopiridol on 30-minute bolus followed by 4-hour infusion.

FIG. 2 shows Assessment of change in 1) expression of anti-apoptotic proteins Mcl-1, XIAP, Bcl-2, and Bax and 2) RNA Polymerase II serine 5 phosphorylation and total RNA Polymerase II protein levels in primary CLL cells during treatment in a representative responding and non-responding patient.

### DETAILED DESCRIPTION OF THE INVENTION

The terms as used herein have the following meanings:

As used herein, “patient” means a warm blooded animal, such as for example rat, mice, dogs, cats, guinea pigs, and primates such as humans.

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier is pharmaceutically acceptable oil typically used for parenteral administration.

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, sulfuric acid, methanesulfonic acid, 2-hydroxyethanesulfonic acid, p-toluenesulfonic acid, fumaric acid, maleic acid, hydroxymaleic acid, malic acid, ascorbic acid, succinic acid, glutaric acid, acetic acid, salicylic acid, cinnamic acid, 2-phenoxybenzoic acid, hydroxybenzoic acid, phenylacetic acid, benzoic acid, oxalic acid, citric acid, tartaric acid, glycolic acid, lactic acid, pyruvic acid, malonic acid, carbonic acid or phosphoric acid. The acid metal salts such as sodium monohydrogen orthophosphate and potassium hydrogen sulfate can also be formed. Also, the salts so formed may present either as mono- or di-acid salts and can exist substantially anhydrous or can be hydrated. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts, and salts formed with suitable organic ligands, e.g. quaternary ammonium salts.

“Therapeutically effective amount” means an amount of the compound which is effective in treating the named disease, disorder or condition.

The term “treating” refers to:
(i) preventing a disease, disorder or condition from occurring in a patient that may be predisposed to the disease, disorder and/or condition, but has not yet been diagnosed as having it;
(ii) inhibiting the disease, disorder or condition, i.e., arresting its development; and
(iii) relieving the disease, disorder or condition, i.e., causing regression of the disease, disorder and/or condition.

As noted above, flavopiridol is a broad cyclin-dependent kinase inhibitor (see generally, de Azevedo W. F., Jr., et al., “Structural basis for inhibition of cyclin-dependent kinase 9 by flavopiridol,” Biochem Biophys Res. Commun. 2002; 293:566-71) that effectively induces apoptosis in both CLL cell lines and human CLL cells in vitro at concentrations attainable in the clinic. See, for example, Byrd J. C., et al., “Flavopiridol induces apoptosis in chronic lymphocytic leukemia cells via activation of caspase-3 without evidence of bcl-2 modulation or dependence on functional p53,” Blood Vol. 92, 1998:3804-3816. Importantly, this anti-tumor activity was observed to occur in a p53-independent manner. Flavopiridol also decreases in the levels of Mcl-1 and XIAP, proteins that mediate resistance to apoptosis in CLL cells in vitro. These studies suggest that flavopiridol may be effective in treating CLL in a human.

However, despite the promising pre-clinical results with flavopiridol in CLL and virtually all hematologic malignancies, the efficacy of flavopiridol in treating CLL in all of the clinical studies reported thus far are disappointing. No activity was seen utilizing a 72-hour continuous infusion and 24-hour infusion, and only a 11% response rate was achieved with a 1-hour bolus in relapsed CLL. See for example, Byrd J. C. et al., “Treatment of Relapsed Chronic Lymphocytic Leukemia by 72-hour Continuous Infusion or 1-Hour Bolus Infusion of Flavopiridol: Results from Cancer and Leukemia Group B Study 19805,” Clin. Cancer Res. 2005;11(11) Jun. 1, 2005, 4176-4181, and references cited therein. The schedules selected in these unsuccessful trials were modeled after in vitro studies performed in fetal bovine serum-based media. Following completion of these preliminary CLL studies, it was noted that the high protein binding of flavopiridol in human serum did not occur with fetal bovine serum. In addition, the data showed that the concentration of flavopiridol required to induce apoptosis of CLL cells in human serum in vitro was substantially higher than that required in fetal bovine serum.

Thus in accordance of this invention a modified dosage regimen was developed that will provide a target concentration in vivo which would alleviate the deficiencies observed in the previous studies.

Surprisingly, it was found that a dosage regimen that provides a gradual dose-escalation of flavopiridol provides remarkable efficacy in treating CLL. Thus in accordance of this invention there is provided a dosing regimen comprising:
b) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
b) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

As disclosed hereinbelow in specific clinical trials a dramatic activity of flavopiridol, a cyclin-dependent kinase inhibitor, is observed in genetically high risk, fludarabine-refractory CLL. Remarkably, the dose limiting toxicity of administering flavopiridol to patients in this trial was acute tumor lysis syndrome (TLS), indicating significant anti-tumor activity. This toxicity is only rarely observed in CLL, and then generally only in the initial treatment of this disease. See, Cheson B. D., et al., “Tumor lysis syndrome: an uncommon complication of fludarabine therapy of chronic lymphocytic leukemia,” J. Clin. Oncol. 1998; 16:2313-20. This observation of dramatic responses, including acute TLS, in patients with refractory CLL indicates substantial potential for this agent in the initial treatment of CLL as part of combination based therapies and as a single agent in patients with high risk genetic features or with refractory disease. Additionally, it suggests that flavopiridol may have similar activity in other B-cell malignancies such as low-grade lymphoma and mantle cell lymphoma, in which similar treatment response profiles with other agents are often observed.

The development of flavopiridol in CLL exemplifies the importance of continued translational investigation throughout the entire clinical development of an agent and adaptation of models that best approximate the in vivo human setting. Despite the remarkable activity of flavopiridol in vitro with both short and long exposures in a broad range of malignancies, only minimal activity has been observed in clinical trials for CLL, related B-cell malignancies, or solid tumors. Specifically, no patients with CLL responded when treated with 72- and 24-hour continuous infusion of flavopiridol, and only 11% responded to the 1-hour bolus schedule. Pharmacokinetic data from the 72-hour and 24-hour continuous infusion studies and 1-hour bolus studies done by others attained plasma drug concentrations that were sufficient to induce apoptosis in vitro in primary CLL cells and other malignancies. One explanation for the lack of concordance between the promising in vitro results and disappointing clinical results may be due to increased flavopiridol protein binding in human serum relative to the fetal calf serum typically used for in vitro studies in pre-clinical investigation.

Based upon the poor clinical results in all the trials reported to date with flavopiridol, the present invention contemplated the possibility that differential protein binding could exist between these two culture media. Surprisingly, in accordance with the practice of this invention it was demonstrated that substitution of human serum for fetal calf serum resulted in a decrease in free drug level from 63-100% to 5-8%, with an increase in 1-hour and 24-hour LC₅₀ values required to induce apoptosis in CLL cells from 670 nM and 120 nM to 3510 nM and 470 nM, respectively. This increase in the in vitro LC₅₀ may be critical, as the 24 hr LC₅₀ of 470 nM has not been achieved in vivo with the 72-hour continuous infusion schedule. Additionally, the 1-hour LC₅₀ concentration for CLL cells in human serum was generally not obtained in the solid tumor phase I trial exploring this schedule. Thus, the prolonged continuous infusion or bolus dosing schedules previously explored in CLL and NHL may not achieve pharmacologically effective drug concentrations of flavopiridol, thereby explaining the absence of activity in the continuous infusion schedules and the marginal activity with the IV bolus schedule. Given the novel mechanism of action of flavopiridol, as well as its ability to induce apoptosis independent of p53 dysfunction and modulate critical anti-apoptotic proteins such as Mcl-1 in vitro, and utilizing pharmacokinetic modeling a new dosing regimen in accordance with this invention was developed.

Thus in one aspect of this dosing regimen a 30-minute bolus followed by a 4-hour continuous infusion schedule could attain the concentration of flavopiridol sufficient to induce apoptosis in primary CLL cells incubated in human plasma in vitro.

In another embodiment of this invention, the dosing regimen is comprised of from about forty percent to about fifty percent of the total therapeutically effective amount of flavopiridol for administering the bolus infusion.

In another embodiment the dosage level of the initial bolus infusion is from about 20 mg/m² to about 50 mg/m². Preferably, the dosage level of the initial bolus infusion is about 30 mg/m² and more preferably the dosage level is about 40 mg/m².

In another embodiment of this invention, the dosing regimen is comprised of from about fifty percent to about sixty percent of the total therapeutically effective amount of flavopiridol for the subsequent infusion.

In another embodiment of this invention, the dosage level of the subsequent infusion is from about 20 mg/m² to about 60 mg/m². Preferably, the dosage level of said infusion is about 30 mg/m². More preferably the dosage level of the infusion is about 40 mg/m² and even more preferably the dosage level of the infusion is about 50 mg/m².

In another embodiment of this invention, the initial bolus infusion can be carried out for a suitable period of time sufficient to reach about half the toxic level as discussed above. Generally, such a period can range from about 30 minutes to about 1 hour. In a further aspect of this embodiment, the bolus infusion is carried out for a period of from about 20 minutes to about 1 hour. In yet another aspect of this embodiment, the bolus infusion is carried out for a period of about 30 minutes.

In yet another embodiment of this invention, the subsequent infusion is generally carried out for a period sufficient to reach the therapeutic efficacy as described hereinabove and such a period is generally about 4 hours. However, any period lesser or larger than this length of period depending upon the patient does not depart from the practice of this invention.

In yet another embodiment, the dosing regimen of this invention is suitable for the treatment of cancer selected from the group consisting of chronic lymphocytic leukemia (CLL), acute lymphocytic leukemia (ALL), lymphoma, acute leukemia, solid tumors, esophageal cancer, breast cancer, lung cancer and prostate cancer.

In yet another aspect of this invention there is provided a method of treating a cancer selected from the group consisting of chronic lymphocytic leukemia (CLL), acute lymphocytic leukemia (ALL), lymphoma, acute leukemia, esophageal cancer, solid tumors, breast cancer, lung cancer and prostate cancer in a patient comprising administering to said patient a dosing regimen comprising:
a) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
b) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

In an embodiment of this aspect of the invention, the bolus infusion as well as the subsequent infusion of flavopiridol can be administered by any of the procedures known in the art. For instance and as discussed in more detail below, the bolus infusion followed by the infusion is administered intravenously.

In this aspect of the practice of the method of this invention all of the specific features described hereinabove for the dosing regimen can be used for the practice of the method of this invention, for example, same percent levels of bolus and subsequent infusion dosage levels, etc. can be used.

In yet another aspect of this invention there is also provided a pharmaceutical kit comprising:
a) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
b) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

The dosing regimen of this invention is in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Flavored unit dosage forms contain from 1 to 100 mg, for example 1, 2, 5, 10, 25, 50 or 100 mg, of the active ingredient. The tablets or pills of the dosage regimen can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the dosing regimen of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The dosing regimen of this invention can be administered by any of the methods known in the art. In general, the dosing regimen of this invention can be administered by oral, intravenous, intramuscular, subcutaneous, rectal, intratracheal, intranasal, intraperitoneal or topical route. The preferred administration of the dosing regimen of this invention is intravenously. Any of the known methods to administer pharmaceutical compositions by intravenous route can be used to administer the dosing regimen of this invention.

This invention is further illustrated by the following examples which are provided for illustration purposes and in no way limit the scope of the present invention.

### EXAMPLE 1

Subjects: All patients provided written informed consent. Patients had CLL or small lymphocytic lymphoma and required therapy according to the NCI criteria. All patients had received at least one prior chemotherapy regimen, although most were fludarabine-refractory as defined previously. Enrollment requirements included: age older than 17 years, symptomatic by the NCI 96 criteria, platelet count greater than 49×10⁹/L, ECOG performance status of 2 or less, no active infection or inflammatory bowel disease, and not pregnant. The serum creatinine and total bilirubin levels were required to be no more than 2.0 times the normal value.

Design, Treatment and Dose Modifications: This study utilized a modified phase I design, enrolling three to six patients per level until two of the first six patients experienced dose limiting toxicity. Expansion of the dose level below this occurred to a minimum of six patients to assure safety for phase II investigation. Flavopiridol was administered via a central venous catheter. The schedule of flavopiridol included receiving 50% of the dose as a 30-minute bolus infusion and the remaining 50% dose as a 4-hour infusion every week for four consecutive weeks, followed by two weeks off. The total dose administered in level 1 was 60 mg/m² (i.e. 30 mg/m² as 30 minute bolus followed by 30 mg/m² 4-hour continuous infusion). In level 2 the dose was 80 mg/m² (i.e. 40 mg/m² bolus and 40 mg/m² infusion). All patients received allopurinol 300 mg/day beginning one day prior to initiation of treatment and continued during therapy. All patients received prophylactic anti-viral and Pneumocystis carinii pneumonia therapy as described previously during and for six months following completion of therapy. Following dose limiting acute TLS in cohort 2, the remaining patients treated at dose level 1 received inpatient pre-treatment hydration and urine alkalization, prophylactic phosphate-binder treatment, and hourly potassium monitoring during and for four hours after treatment. Aggressive management of hyperkalemia including kayexalate therapy, insulin and glucose therapy, and calcium therapy was employed according to an established protocol. The ability to perform bedside emergent dialysis was assured. Prophylactic rasburicase was utilized in patients with bulky lymphadenopathy or high lymphocyte counts. Following five treatments with flavopiridol, treatment was transitioned to outpatient status with two hours of hydration before and through therapy with abbreviated laboratory monitoring. Patients could discontinue therapy for stable disease without improvement after two cycles (eight treatments). Otherwise, patients continued therapy in the absence of progression or toxicity that prohibited further treatment for a maximum of six cycles (24 treatments) of flavopiridol. Patients were evaluated for response after two, four, and six cycles of therapy.

Dose Limiting Toxicity: Dose limiting toxicity was defined as non-hematologic toxicity of grade 3 or greater severity (excluding transient liver function abnormalities, transient non-life-threatening electrolyte abnormalities, fatigue, or diarrhea that resolve within four days), or in some case grade 2 toxicity (i.e. irreversible renal, chronic pulmonary, neurologic, or cardiac toxicity). Hematologic toxicity was dose limiting only if grade 4 thrombocytopenia or neutropenia persisted for seven days or greater. The NCI Common Toxicity Criteria was used to grade non-hematologic toxicity and the NCI 96 CLL criteria for hematologic toxicity. See, Cheson B. D., et al. “National Cancer Institute-sponsored Working Group guidelines for chronic lymphocytic leukemia: revised guidelines for diagnosis and treatment,” Blood 1996; 87:4990-7.

Assessment of Response: Criteria for response and progression utilized the Revised NCI-sponsored Working Group Guidelines as mentioned above. As specified by these guidelines, a response had to be maintained for a period of two months.

Molecular and Cellular Pharmacodynamic Studies: Baseline interphase cytogenetic studies and p53 mutational studies were performed as previously published (Lozanski G. H. N., et al., “Alemtuzumab is an Effective Therapy for Chronic Lymphocytic Leukemia with p53 Mutations and Deletions,” Blood 2004; 103:3278-81). Serial tumor cell samples at baseline, four hours into infusion, and 24 hours post-infusion were obtained and cellular protein lysates made as previously described. Immunoblotting for Mcl-1, XIAP, Bax, Bcl-2, and RNA polymerase II phosphorylation was performed using previously published methods (Parker B. W., et al., “Early induction of apoptosis in hematopoietic cell lines after exposure to flavopiridol,” Blood Vol. 91, 1998:458-465). Sources of antibodies used were: XIAP (BD Biosciences, San Jose Calif.), Mcl-1, Bcl-2 and Bax (Santa Cruz Biotechnology, Santa Cruz Calif.), RNA Polymerase II, phosphoserine and RNA Polymerase II, total (Covance Research Products, Berkeley Calif.), Glyceraldehyde 3-Phosphate Dehydrogenase (GAPDH) (Chemicon, Temecula Calif.).

Pharmacokinetics: Plasma samples for pharmacokinetics were obtained prior to dosing (t=0), and at 30 minutes (0.5 hour), 4.5, 6, 8, 12, 18, 24 and 32 hours after the start of the bolus infusion, and analyzed utilizing a liquid chromatographic/mass spectrometric (LC/MS) assay. An aliquot of each plasma sample was spiked with internal standard, extracted with ethyl acetate, and the organic phase was evaporated under nitrogen gas. The residue was reconstituted with the initial mobile phase and injected into LC/MS. Analytes were separated using a C18 Polaris column and an aqueous mobile phase containing 25 mM ammonium acetate, pH 4.15) with the percentage of methanol increased from 40% to 90% during each chromatographic run. Flavopiridol (401 m/z) was detected using electrospray ionization in the positive ion mode on an Agilent System 1100 mass selective detector (MSD). The limit of quantitation of this assay was 5 nM. Within- and between-day precision was within 12.4% to 19.6% coefficient of variation.

Patient Characteristics: A total of 23 patients were enrolled and treated on this study on dose level 1 (n=20 patients) and dose level 2 (n=3 patients). The demographics of these patients are shown in Table 1. These patients had a median number of therapies of four (range 2-13) with 91% of these being refractory to their last treatment. Seventeen (74%) patients had high risk genetic features [del(17p13.1), del(11q22.3), or complex karyotype (greater than or equal to three abnormalities] at pre-treatment evaluation.

TABLE 1
Pre-treatment Patient Characteristics

| Median Age | 61 (range 44-84) |
|---|---|
| Stage | |
| Rai Intermediate Risk | 39% |
| Rai High Risk | 61% |
| % Female | 35% |
| Performance Status | |
| 0 | 22% |
| 1 | 56% |
| 2 | 22% |
| Median Number Therapies | 4 (range 2-13) |
| del(11q22.3) | 39% |
| del(17p13.1) or p53 mutation | 39% |
| Complex karyotype | 35% |
| Complex, del(11q22.3), del(17p13.1) | 74% |
| Median leukocyte count (×10⁹/L) | 8.3 (range 3.1-200) |
| Median hemoglobin (mmol/L) | 10.1 (range 6.5-15.4) |
| Median platelets (×10⁹/L) | 117 (range 53-254) |
| Patients with splenomegaly | 39% |
| Patients with adenopathy | 100% |
| Patients with bulky adenopathy (>5 cm) | 57% |

Toxicity Observed with Flavopiridol: Six patients were initially treated in dose level 1 (30 mg/m² bolus followed by 30 mg/m² 4-hour infusion), with one patient having dose limiting toxicity (neutropenic fever). Dose escalation proceeded to dose level 2, with three patients being treated. Patient two at this dose level experienced dose-limiting toxicity of acute TLS requiring a two-day hospitalization but not dialysis and attained a durable (>13 month) partial response. Patient three at dose level 2 developed TLS complicated by uncontrollable hyperkalemia and subsequent fatal asystole on day 1 before dialysis could be initiated. On autopsy, extensive apoptosis/necrosis of diffuse intra-abdominal lymphadenopathy was found. No additional patients were treated at this dose, but patient one of dose level 2, who previously had not experienced TLS at the 80 mg/m²/dose, developed this toxicity on day 1 of cycle 2 while receiving a 60 mg/m² dose. All treatment was put on hold and an aggressive monitoring mechanism was put into place. Fourteen additional patients were treated at the level 1 dose utilizing this protocol. While biochemical evidence of TLS was present in most patients receiving therapy, only one developed significant TLS that required temporary dialysis for medically uncontrollable hyperkalemia on day 1 of therapy. Other grade 3 and 4 toxicities observed with flavopiridol treatment are summarized in Table 2. While grade 1 (n=11) or 2 (n=6) diarrhea occurred in the majority of patients treated at the level 1 dose, this was generally short-lived. Similarly, transient grade 1 (n=13) or grade 2 (n=6) fatigue was observed.

TABLE 2
Number and Percentage of Patients Experiencing Grade 3 or Greater
Toxicities During and After Treatment with Flavopiridol

| | Toxicity | | |
|---|---|---|---|
| | Grade 3 | Grade 4 | Grade 5 |
| Neutropenia | 3 (13%) | 20 (87%) | 0 |
| Thrombocytopenia | 3 (13%) | 3 (13%) | 0 |
| Anemia | 6 (26%) | 0 | 0 |
| Infection | 5 (22%) | 0 | 0 |
| Tumor Lysis Syndrome | 3 (13%) | 0 | 1 (4%) |
| Hypokalemia | 3 (13%) | 0 | 0 |
| Syncope | 3 (13%) | 0 | 0 |
| Diarrhea | 3 (13%) | 0 | 0 |
| Arthralgias | 0 | 1 (4%) | 0 |
| Edema | 1 (4%) | 0 | 0 |
| Fatigue | 1 (4%) | 0 | 0 |
| Dyspnea | 1 (4%) | 0 | 0 |
| AST Elevation | 1 (4%) | 0 | 0 |

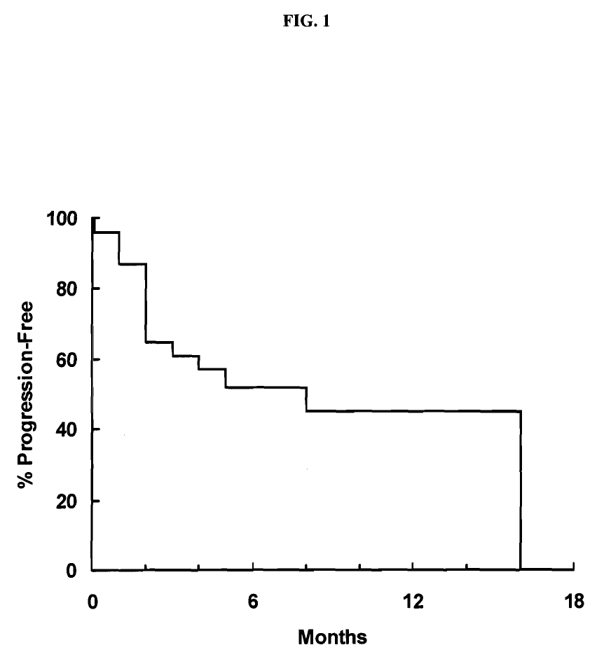

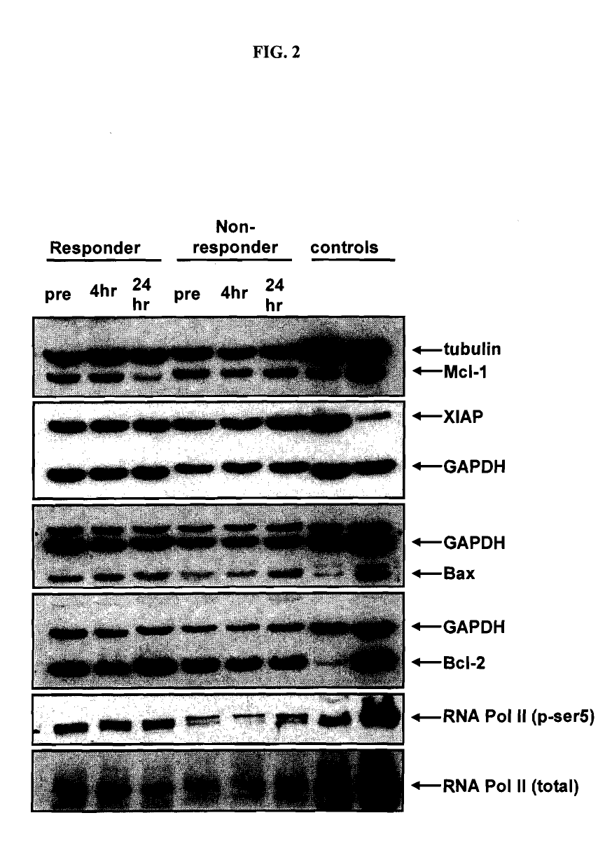

Response to Treatment and Characteristics Associated with Response: All patients were evaluable for response. Among the 23 patients enrolled on the trial, ten (43%) attained a partial response as defined by the NCI 96 criteria. Patients received a median of 2 cycles (range 1-6). Response was generally rapid, with eight of the nine patients with measurable lymphadenopathy experiencing a 50% or greater reduction in lymphadenopathy by day 8 of cycle 1. Eight of ten patients remain in continuous remission at five to 14+ months, as depicted in FIG. 1, while two patients relapsed at eight and sixteen months. Characteristics of the responding patients were examined. Eight of the ten responding patients had fludarabine-refractory disease and lymph node size greater than 5 centimeters, a parameter associated with poor response to alemtuzumab. Nine of ten patients had high risk genetic features including del(17p13.1) [n=3] or del(11q22.3) [n=7] that predict poor response to traditional CLL therapies.
Cellular Pharmacodynamic Studies: As flavopiridol has been observed to promote down-modulation of proteins such as Mcl-1 and XIAP in vitro in primary CLL cells possibly through inhibiting RNA polymerase II and thus global gene transcription, it was examined if this occurs in vivo in a subset of eight patients who had high circulating blood lymphocyte counts. None of the five non-responding patients had significant modulation of Mcl-1, while two of three responding patients examined demonstrated decreased expression of this protein with treatment. The mechanism of this Mcl-1 down-regulation did not appear to be secondary to inhibition of CDK9 and subsequent diminishment of serine-5 phosphorylation of RNA polymerase II, as no change in this parameter was observed in any of the eight patients examined. A representative immunoblot of a responding and non-responding patient is included in FIG. 2.

Pharmacokinetic Studies Flavopiridol plasma concentrations declined in a biexponential manner after the end of the 4-hour CIVI with a terminal half-life of 1.1 to 42.5 hr (14.0±10.2 hr; mean ±S.D.), and flavopiridol plasma concentrations were detectable throughout the study using the LC/MS method described above. Pharmacokinetic parameters of flavopiridol in this trial are summarized in Table 3. Flavopiridol exhibited significant inter-patient variability, with Cₘₐₓ varying two to three-fold at each dose level. The mean CL of flavopiridol was 14.5±6.5, similar to that reported in prior studies. While these studies were conducted in all patients, the limited size of the overall phase I study prevented identification of statistically significant correlation between the occurrence of TLS and Cₛₛ during infusion or area under the curve (AUC). However, patient 9 treated on dose level 2, who died from overwhelming tumor lysis, had the highest observed AUC during the infusions and the second highest Cₘₐₓ (4.4 μM) of any of the patients that were evaluated, suggesting that high plasma flavopiridol concentrations and/or exposure (i.e., AUC) may be associated with tumor lysis or clinical activity.

TABLE 3
Flavopiridol Pharmacokinetic Parameters

| | | Mean ± S.D. |
|---|---|---|
| Parameter | Unit | 30 + 30 mg/m² (n = 20) |
| AUC | μM×hr | 10.9 ± 6.3 |
| CL | L/hr/m² | 14.5 ± 6.5 |
| t_{1/2} | Hr | 14.0 ± 10.2 |
| V_{dβ} | L/m² | 267 ± 199 |

### EXAMPLE 2

Fifty-six patients were enrolled in this clinical trial with the median prior therapies being 4 and the majority being fludarabine-refractory. The dose limiting toxicity in cohort 2 was tumor lysis syndrome (TLS). Cohort 1 was expanded with aggressive TLS prophylaxis. Of the 20 patients in cohort 1, 8 (40%) attained a partial response (PR) with median response duration exceeding 12 months. The 0.5 and 4.5 hr Cₘₐₓ were 2.08 μM and 0.96 μM, respectively. PK modeling demonstrated increasing the 4-hr infusion would increase the 4.5 hr Cₘₐₓ to the desired level. Cohort 3 and 4 did this (Table 4) with acceptable toxicity. Cohort 3 included 19 patients of whom 14 were escalated with the 5^{th} dose. Increased tumor cytoreduction was observed in a majority of patients with dose escalation. Ten (53%) of patients attained a partial response. The 0.5 and 4.5 hr Cₘₐₓ was 1.95 μM and 1.54 μM at the escalated doses for patients in cohort 3. TLS risk factors were examined for the first treatment dose in cohorts 1-3. WBC of ≧200×10⁹/L were more frequently associated with TLS (5 of 8 pts) versus those with WBC<200×10⁹/L (1 of 34 pts). Cohort 4 excludes pts with WBC≧200×10⁹/L and currently includes 14 pts. Safety has been acceptable with only 1 case of TLS. Response assessment for cohort 4 is underway, with anti-tumor activity appearing similar to that observed in cohort 3.

Conclusions: Flavopiridol with this PK directed schedule has significant clinical activity independent of the presence of del(17p13.1) and represents one of he most active agents tested for the treatment of CLL.

TABLE 4

| | | No | TLS Dose | Response |
|---|---|---|---|---|
| Cohort | Dosing Schedule | Pts | 1* | Rate |
| 1 | 30 mg/m² + 30 mg/m² | 20 | 2 (10%) | 8 (40%) |
| 2 | 40 mg/m² + 40 mg/m² | 3 | 1 (33%) | 1 (33%) |
| 3 | 30 mg/m² + 30 mg/m² dose 1-4 | 19 | 4 (22%) | 10 (53%) |
| | followed by 30 mg/m² + 50 mg/m² | | | |
| | dose 5 and hereafter | | | |
| 4 | 30 mg/m² + 30 mg/m² dose 1 | 14 | 1 (7%) | Pending |
| | followed by 30 mg/m² + 50 mg/m² | | | |
| | dose 2 and thereafter | | | |

*Defined as TLS requiring dialysis;
Cohort 4: Escalate dose to 30 mg/m² + 50 mg/m² with dose 5;
Cohort 4 escalate with dose 2

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A dosing regimen comprising:
c) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
d) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

2. The dosing regimen as set forth in claim 1, wherein said regimen is comprised of from about forty percent to about fifty percent of the total therapeutically effective amount of flavopiridol for said bolus infusion.

3. The dosing regimen as set forth in claim 1, wherein the dosage level of said bolus infusion is from about 20 mg/m² to about 50 mg/m².

4. The dosing regimen as set forth in claim 3, wherein the dosage level of said bolus infusion is about 30 mg/m².

5. The dosing regimen as set forth in claim 3, wherein the dosage level of said bolus infusion is about 40 mg/m².

6. The dosing regimen as set forth in claim 1, wherein said regimen is comprised of from about fifty percent to about sixty percent of the total therapeutically effective amount of flavopiridol for said infusion.

7. The dosing regimen as set forth in claim 1, wherein the dosage level of said infusion is from about 20 mg/m² to about 60 mg/m².

8. The dosing regimen as set forth in claim 7, wherein the dosage level of said infusion is about 30 mg/m².

9. The dosing regimen as set forth in claim 7, wherein the dosage level of said infusion is about 40 mg/m².

10. The dosing regimen as set forth in claim 7, wherein the dosage level of said infusion is about 50 mg/m².

11. The dosing regimen as set forth in claim 1, wherein said bolus infusion is carried out for a period of from about 30 minutes to about 1 hour.

12. The dosing regimen as set forth in claim 1, wherein said bolus infusion is carried out for a period of from about 20 minutes to about 1 hours.

13. The dosing regimen as set forth in claim 1, wherein said bolus infusion is carried out for a period of about 30 minutes.

14. The dosing regimen as set forth in claim 1, wherein said infusion is carried out for a period of about 4 hours.

15. The dosing regimen as set forth in claim 1, wherein said regimen is suitable for the treatment of cancer selected from the group consisting of chronic lymphocytic leukemia (CLL), acute lymphocytic leukemia (ALL), lymphoma, acute leukemia, solid tumors, esophageal cancer, breast cancer, lung cancer and prostate cancer.

16. The dosing regimen as set forth in claim 1, wherein said regimen is suitable for the treatment of chronic lymphocytic leukemia (CLL).

17. The dosing regimen as set forth in claim 1, wherein said regimen is suitable for the treatment of lymphoma.

18. The dosing regimen as set forth in claim 1, wherein said regimen is suitable for the treatment of acute leukemia.

19. The dosing regimen as set forth in claim 1, wherein said regimen is suitable for the treatment of solid tumors.

20. The dosing regimen as set forth in claim 1, wherein said regimen is suitable for the treatment of breast cancer.

21. The dosing regimen as set forth in claim 1, wherein said regimen is suitable for the treatment of lung cancer.

22. The dosing regimen as set forth in claim 1, wherein said regimen is suitable for the treatment of prostate cancer.

23. A method of treating a cancer selected from the group consisting of chronic lymphocytic leukemia (CLL), acute lymphocytic leukemia (ALL), lymphoma, acute leukemia, esophageal cancer, solid tumors, breast cancer, lung cancer and prostate cancer in a patient comprising administering to said patient a dosing regimen comprising:
a) a therapeutically effective amount of flavopiridol or a pharmaceutically acceptable salt thereof optionally in combination with a pharmaceutically acceptable carrier suitable for bolus infusion over a period of from about 15 minutes to about 2 hours; and
b) a therapeutically effective amount of flavopiridol or a pharmaceutically acceptable salt thereof optionally in combination with a pharmaceutically acceptable carrier suitable for a subsequent administration by infusion over a period of from about 4 to about 6 hours.

24. The method as set forth in claim 23, wherein said bolus infusion and said infusion of flavopiridol is administered intravenously.

25. The method as set forth in claim 23, wherein said regimen is comprised of from about forty percent to about fifty percent of the total therapeutically effective amount of flavopiridol for said bolus infusion.

26. The method as set forth in claim 23, wherein said bolus infusion of flavopiridol is administered at a dosage level of from about 20 mg/m² to about 50 mg/m².

27. The method as set forth in claim 26, wherein said bolus infusion of flavopiridol is administered at a dosage level of about 30 mg/m².

28. The method as set forth in claim 26, wherein said bolus infusion of flavopiridol is administered at a dosage level of about 40 mg/m².

29. The method as set forth in claim 23, wherein said regimen is comprised of from about fifty percent to about sixty percent of the total therapeutically effective amount of flavopiridol for said infusion.

30. The method as set forth in claim 23, wherein said infusion of flavopiridol is administered at a dosage level of from about 20 mg/m² to about 60 mg/m².

31. The method as set forth in claim 30, wherein said infusion of flavopiridol is administered at a dosage level of about 30 mg/m².

32. The method as set forth in claim 30, wherein said infusion of flavopiridol is administered at a dosage level of about 40 mg/m².

33. The method as set forth in claim 30, wherein said infusion of flavopiridol is administered at a dosage level of about 50 mg/m².

34. The method as set forth in claim 23, wherein said bolus infusion is carried out for a period of from about 30 minutes to about 1 hour.

35. The method as set forth in claim 23, wherein said bolus infusion is carried out for a period of from about 20 minutes to about 1 hour.

36. The method as set forth in claim 23, wherein said bolus infusion is carried out for a period of about 30 minutes.

37. The method as set forth in claim 23, wherein said infusion is carried out for a period of about 4 hours.

38. The method as set forth in claim 23, wherein said cancer is chronic lymphocytic leukemia (CLL).

39. The method as set forth in claim 23, wherein said cancer is lymphoma.

40. The method as set forth in claim 23, wherein said cancer is acute leukemia.

41. The method as set forth in claim 23, wherein said cancer is solid tumors.

42. The method as set forth in claim 23, wherein said cancer is breast cancer.

43. The method as set forth in claim 1, wherein said cancer is lung cancer.

44. The method as set forth in claim 1, wherein said cancer is prostate cancer.
